(19) United States
(12) Patent Application Publication
(10) Pub. No.: US 2020/0087726 A1
(43) Pub. Date: Mar. 19, 2020

(54) ULTRA SENSITIVE PROBES FOR DETECTION OF NUCLEIC ACID

(71) Applicant: QuanDx Inc., San Jose, CA (US)

(72) Inventors: Xiaojun LEI, San Jose, CA (US); Yuan YUAN, San Jose, CA (US); Qiang LI, San Jose, CA (US); Yi ZHANG, Wilmette, IL (US)

(21) Appl. No.: 16/701,119

(22) Filed: Dec. 2, 2019

Related U.S. Application Data
(63) Continuation-in-part of application No. 15/573,131, filed on Nov. 10, 2017, now abandoned.
(60) Provisional application No. 62/159,318, filed on May 10, 2015.

(51) Int. Cl. C12Q 1/6876; C12Q 1/6818; C12Q 1/6834

(52) U.S. Cl. CPC C12Q 1/6876; C12Q 1/6818; C12Q 1/6834; C12Q 2525/197; C12Q 2565/549; C12Q 2565/101; C12Q 2565/30; C12Q 2565/507; C12Q 2525/185

(57) ABSTRACT

The present disclosure provides a composition with ultra sensitivity for detection of nucleic acid and the method of use thereof. The composition comprises a target probe capable of hybridizing to a target nucleic acid, at least one first bridge probe, at least one second bridge probe, and a label probe. The target probe includes a pre-bridge region having a first tail nucleotide sequence. The first bridge probe includes sequentially a first head region having a first head nucleotide sequence, a first gap region having a gap nucleotide sequence, and a first tail region having the first tail nucleotide sequence. The second bridge probe includes sequentially a second head region having a second head nucleotide sequence complementary to the first head nucleotide sequence, a second gap region having the gap nucleotide sequence, and a second tail region having a second tail nucleotide sequence complementary to the first tail nucleotide sequence. The label probe is capable of hybridizing to the first and the second gap nucleotide region.

### CROSS-REFERENCE TO RELATED APPLICATIONS

This application is a continuation-in-part of U.S. application Ser. No. 15/573,131, which is a national phase of PCT/US2016/031552 filed May 10, 2016, which claims priority to U.S. provisional patent application No. 62/159,318 filed May 10, 2015, the entire disclosure of which is incorporated herein by reference.

### FIELD OF THE INVENTION

The present invention generally relates to nucleic acid chemistry and assays. More particularly, the invention relates to probes and methods for detection of nucleic acid in a sample.

### BACKGROUND OF THE INVENTION

Hybridization-based methods for detection of nucleic acid, such as Northern and Southern blot, in situ hybridization, have broad application in molecular diagnostic and biomedical research. In principle, a hybridization probe is generated by conjugating a label that provides detectable signal, such as radioactivity and fluorescence, to a fragment of DNA or RNA with sequence complementary to a target sequence. The hybridization probe hybridizes to single-stranded nucleic acid (DNA or RNA) containing the target sequence due to complementarity between the probe and target. The signal from the label is detected to determine the presence or absence of the target sequence.

However, the application of hybridization probe can be limited by its inability to detect DNA or RNA targets with low copy numbers due to lack of sensitivity and specificity. Therefore, there is continuing need to develop probes with ultra sensitivity to detect nucleic acids.

### BRIEF SUMMARY OF THE INVENTION

In one aspect, the present disclosure provides a composition comprising a target probe capable of hybridizing to a target nucleic acid, at least one first bridge probe, at least one second bridge probe, and at least one label probe having a label. The target probe includes a pre-bridge region having a first tail nucleotide sequence. The first bridge probe includes sequentially a first head region having a first head nucleotide sequence, a first gap region having a gap nucleotide sequence, and a first tail region having the first tail nucleotide sequence. The second bridge probe includes sequentially a second head region having a second head nucleotide sequence complementary to the first head nucleotide sequence, a second gap region having the gap nucleotide sequence, and a second tail region having a second tail nucleotide sequence complementary to the first tail nucleotide sequence. The label probe is capable of hybridizing to the first and the second gap nucleotide region.

In certain embodiments, the first and second head nucleotide sequence consists of 4-20 nucleotides (e.g., 4, 5, 6, 7, 8, 9, 10, 11, 12, 13, 14, 15, 16, 17, 18, 19 or 20 nucleotides).

In certain embodiments, the first and second tail nucleotide sequence consists of 4-20 nucleotides (e.g., 4, 5, 6, 7, 8, 9, 10, 11, 12, 13, 14, 15, 16, 17, 18, 19 or 20 nucleotides).

In certain embodiments, the gap nucleotide sequence consists of 6-20 nucleotides (e.g., 6, 7, 8, 9, 10, 11, 12, 13, 14, 15, 16, 17, 18, 19 or 20 nucleotides).

In certain embodiments, the label is a fluorophore, a horse radish peroxidase or an alkaline phosphatase.

In certain embodiments, the probe (e.g., the target probe, bridge probe, label probe) described herein can comprise one or more nucleotide analogs (e.g., altered backbone, sugar, or nucleobase). In certain embodiments, the nucleotide analog is selected from the group consisting of 5-bromouracil, a peptide nucleic acid nucleotide, a xeno nucleic acid nucleotide, a morpholino, a locked nucleic acid nucleotide, a glycol nucleic acid nucleotide, a threose nucleic acid nucleotide, a dideoxynucleotide, a cordycepin, a 7-deaza-GTP, a fluorophore (e.g. rhodamine or flurescein linked to the sugar), a thiol containing nucleotide, a biotin linked nucleotide, a fluorescent base analog, a methyl-7-guanosine, a methylated nucleotide, an inosine, thiouridine, a pseudourdine, a dihydrouridine, a queuosine, and a wyosine. In certain embodiments, the nucleotide analog is a locked nucleic acid nucleotide.

In certain embodiments, the target nucleic acid is selected from the group consisting of a DNA, a cDNA, a RNA, a mRNA, a rRNA, a miRNA, a Lnc RNA and a siRNA. In certain embodiments, the target nucleic acid is a single-stranded DNA or RNA.

In certain embodiments, the target probe further comprises a label region having the gap nucleotide sequence.

In certain embodiments, the composition described above further comprises a capture probe capable of hybridizing to the target nucleic acid, said capture probe having a magnetic bead or a biotin.

In one embodiment, the first bridge probe is the same as the second bridge probe.

In one embodiment, the present disclosure provides a composition comprising a target probe capable of hybridizing to a target nucleic acid, at least one bridge probe and a label probe capable of hybridizing to the bridge probe. The target probe comprises a pre-bridge region having a tail palindromic sequence. The bridge probe comprises sequentially (i) a first region having a head palindromic sequence; (ii) a second region having a gap sequence; and (iii) a third region having the tail palindromic sequence. The label probe comprises a label.

In certain embodiments, the head palindromic sequence consists of 4-20 nucleotides (e.g., 4, 5, 6, 7, 8, 9, 10, 11, 12, 13, 14, 15, 16, 17, 18, 19 or 20 nucleotides).

In certain embodiments, the tail palindromic sequence consists of 4-20 nucleotides (e.g., 4, 5, 6, 7, 8, 9, 10, 11, 12, 13, 14, 15, 16, 17, 18, 19 or 20 nucleotides).

In another aspect, the present disclosure provides a method of detecting a target nucleic acid in a sample. According to one embodiment, the method comprises the steps of: a) contacting a target probe to the target nucleic acid; b) associating a first bridge probe, a second bridge probe and a label probe with the target probe; and c) detecting the presence, absence, or amount of the label probe associated with the target molecule. The target probe is capable of hybridizing to the target nucleic acid and comprises a pre-bridge region having a tail nucleotide sequence. The first bridge probe comprises sequentially (i) a first head region having a first head nucleotide sequence; (ii) a first gap region having a gap nucleotide sequence; and (iii) a first tail region having the tail nucleotide sequence. The second bridge probe comprises sequentially (i) a second head region having a second head nucleotide sequence complementary to the first head nucleotide sequence; (ii) a second gap region having the gap nucleotide sequence; (iii) a second tail region having a second tail nucleotide sequence complementary to the first tail nucleotide sequence. The label probe is capable of hybridizing to the bridge probe and has a label.

In certain embodiments, the sample is selected from the group consisting of sera, plasma, saliva, urine, cell and tissue.

In certain embodiments, the method further comprises the step of capturing said target nucleic acid with a capture probe comprising a magnetic bead or a biotin, wherein the capture probe is capable of hybridizing to the target nucleic acid.

### BRIEF DESCRIPTION OF THE FIGURES

FIG. 1 shows an ultra sensitive probe composition according to an embodiment of the invention.

FIG. 2 shows an ultra sensitive probe composition according to another embodiment of the invention.

FIG. 3 shows the sequences of the target nucleic acid, the capture probe, the target probe, the first bridge probe, the second bridge probe and label probe (Yin-Yang Probe P1) used in Example 1.

FIG. 4 shows a method of detecting a target nucleic acid using an ultra sensitive probe composition according Example 1.

FIG. 5A shows the detection of the target nucleic acid using the probe composition of Example 1.

FIG. 5B shows that both the first bridge probe and the second bridge probe are required for the detection of the target nucleic acid in Example 1.

FIG. 6 shows the sequence of bridge probe having palindromic sequences, which is used to detect the target nucleic acid of Example 2.

FIG. 7 shows the result of a method for detecting a target nucleic acid using a probe composition according to Example 2.

FIG. 8 shows the sequences of the target nucleic acid, the first bridge probe, and the second bridge probe used in Example 3.

FIG. 9 shows the detection of the target nucleic acid using the probe composition of Example 3.

### DETAILED DESCRIPTION OF THE INVENTION

In the Summary of the Invention above and in the Detailed Description of the Invention, and the claims below, and in the accompanying drawings, reference is made to particular features (including method steps) of the invention. It is to be understood that the disclosure of the invention in this specification includes all possible combinations of such particular features. For example, where a particular feature is disclosed in the context of a particular aspect or embodiment of the invention, or particular claim, that feature can also be used, to the extent possible, in combination with and/or in the context of other particular aspects and embodiments of the invention, and in the invention generally.

The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

Where a range of value is provided, it is understood that each intervening value, to the tenth of the unit of the lower limit unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, the embodiments described herein can be practiced without there specific details. In other instances, methods, procedures and components have not been described in detail so as not to obscure the related relevant function being described. Also, the description is not to be considered as limiting the scope of the implementations described herein. It will be understood that descriptions and characterizations of the embodiments set forth in this disclosure are not to be considered as mutually exclusive, unless otherwise noted.

The following definitions are used in the disclosure:

It is understood that as used herein and in the appended claims, the singular forms “a,” “an,” and “the” include the plural reference unless the context clearly dictates otherwise. Thus, for example, a reference to a “bridge probe” is a reference to one or more bridge probes, and includes equivalents thereof known to those skilled in the art and so forth.

As used herein, “associate” or “associating” means physically direct or indirect attachment. For example, the label probe can hybridize to one or more bridge probe, which hybridizes to the target probe, which hybridizes the target nucleic acid, thereby the label probe is associated with the target nucleic acid.

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)−(a second number),” this means a range whose lower limit is the first number and whose upper limit is the second number. For example, 4 to 20 nucleotides means a range whose lower limit is 4 nucleotides, and whose upper limit is 20 nucleotides.

A “bridge probe” as used herein is a nucleic acid that is capable of hybridizing to the target probe and the label probe. The bridge probe is also capable of hybridizing with other bridge probe and forming a ladder-shaped structure, which is capable of hybridizing to multiple label probes, thus amplifying the signal detectable. Typically, the bridge probe includes sequentially a head region having a head nucleotide sequence, a gap region having a gap nucleotide sequence, and a tail region having a tail nucleotide sequence. In certain embodiments, the bridge probe includes sequentially a first palindromic nucleotide sequence (i.e., the head palindromic sequence), a second nucleotide sequence (i.e., the gap nucleotide sequence) that is complementary to a nucleotide sequence of the label probe; and a third palindromic nucleotide sequence (i.e., the tail palindromic sequence). For example, a bridge probe can have a head palindromic sequence 5′-AGCT-3′ and a tail palindromic sequence 5′-GCGC-3′.

As used herein, a “capture probe” refers to a polynucleotide that is capable of hybridizing to the target nucleic acid. Typically, the capture probe includes a nucleotide sequence that has 6-20 nucleotides and is substantially complementary to a sequence of the target nucleic acid. In preferred embodiments, the sequence of the target nucleic acid that is complementary to the capture probe is not overlapping with the sequence complementary to the target probe. In certain embodiments the capture probe is conjugated to a magnetic bead, through which a complex comprising the target nucleic acid and the ultra sensitive composition disclosed herein can be captured.

The term “complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%>, 70%>, 80%>, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%. 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

The term “hybridizing” refers to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all to, other sequences in a mixed population (e.g., a cell lysate or DNA preparation from a tissue biopsy). A “stringent hybridization” and “stringent hybridization wash conditions” in the context of nucleic acid hybridization (e.g., as in array, microarray, Southern or northern hybridizations) are sequence dependent, and are different under different environmental parameters. An extensive guide to the hybridization of nucleic acids is found in, e.g., Tijssen Laboratory Techniques in Biochemistry and Molecular Bio logy—Hybridization with Nucleic Acid Probes part I, Ch. 2, “Overview of principles of hybridization and the strategy of nucleic acid probe assays,” (1993) Elsevier, N.Y. Generally, highly stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for hybridization of complementary nucleic acids which have more than 100 complementary residues on an array or on a filter in a Southern or northern blot is 42° C. using standard hybridization solutions (see, e.g., Sambrook and Russell Molecular Cloning: A Laboratory Manual (3rd ed.) Vol. 1-3 (2001) Cold Spring Harbor Laboratory, Cold Spring Harbor Press, N.Y.). An example of highly stringent wash conditions is 0.15 M NaCl at 72° C. for about 15 minutes. An example of stringent wash conditions is a 0.2×SSC wash at 65° C. for 15 minutes. Often, a high stringency wash is preceded by a low stringency wash to remove background probe signal. An example medium stringency wash for a duplex of, e.g., more than 100 nucleotides, is 1×SSC at 45° C. for 15 minutes. An example of a low stringency wash for a duplex of, e.g., more than 100 nucleotides, is 4×SSC to 6×SSC at 40° C. for 15 minutes.

As used herein, the term “nucleic acid” (interchangeable with the term “polynucleotide”) encompasses any physical string of monomer units that can be corresponded to any physical string of monomer units that can be corresponded to a string of nucleotides, including a polymer of nucleotides (e.g., a typical DNA or RNA polymer), peptide nucleic acids (PNAs), modified oligonucleotides (e.g., oligonucleotides comprising nucleotides that are not conventional to biological RNA or DNA, such as 2′-O-methylated oligonucleotides), and the like. The nucleic acid can be both single-stranded and double-stranded. The nucleotides of the polynucleotide can be deoxyribonucleotides, ribonucleotides or nucleotide analogs, can be natural and unnatural, and can be unsubstituted, unmodified, substituted or modified. The nucleotides can be linked by phosphodiester bonds, or by phosphorothioate linkages, methylphosphonate linkages, boranophosphate linkages, or the like. The polynucleotide can additionally comprise non-nucleotide elements such as labels, quenchers, blocking groups, or the like. The polynucleotide can be, e.g., single-stranded or double-stranded.

As used herein, a “nucleotide analog” refers to a nucleotide (deoxyribonucleotide or ribonucleotide) comprising one or more modifications (e.g. altered backbone, sugar, or nucleobase). Some non-limiting examples of nucleotide analogs include: 5-bromouracil, peptide nucleic acid nucleotides, xeno nucleic acid nucleotides, morpholinos, locked nucleic acid nucleotides, glycol nucleic acid nucleotides, threose nucleic acid nucleotides, dideoxynucleotides, cordycepin, 7-deaza-GTP, florophores (e.g., rhodamine or flurescein linked to the sugar), thiol containing nucleotides, biotin linked nucleotides, fluorescent base analogs, CpG islands, methyl-7-guanosines, methylated nucleotides, inosines, thiouridines, pseudourdines, dihydrouridines, queuosines, and wyosines.

Xeno nucleic acid (XNA) refers to a group of synthetic polymers similar to DNA and RNA that differ in the sugar backbone. Examples of XNA include without limitation 1,5-anhydrohexitol nucleic acid (HNA), cyclohexene nucleic acid (CeNA), Threose nucleic acid (TNA), glycol nucleic acid (GNA), locked nucleic acid (LNA), peptide nucleic acid (PNA).

Peptide nucleic acid (PNA) is an artificial synthesized polymer similar to DNA or RNA. While DNA and RNA have a deoxyribose and ribose sugar backbone, respectively, PNA's backbone is composed of repeating N-(2′aminoethyl)-glycine units linked by peptide bonds. The various purine and pyrimidine bases are linked to the backbone by a methylene bridge (—CH₂—) and a carbonyl group (—(C═O)—). Because the backbone of PNA contains no charged phosphate groups, the binding between PNA/DNA strands is stronger than between DNA/DNA strands due to the lack of electrostatic repulsion, resulting in increased melting temperature.

A locked nucleic acid is a modified RNA nucleotide whose ribose moiety is modified with an extra bridge connecting the 2′ oxygen and 4′ carbon. The bridge “locks” the ribose in the 3′-endo conformation, which enhances base stacking and backbone pre-organization of the locked nucleic acid, thus significantly increases its hybridization properties (melting temperature).

Threose nucleic acid (TNA) has a backbone structure composed of repeating threose sugars linked together by phosphodiester bonds. TNA can self-assemble by Wastson-Crick base pairing into duplex structure and can form base pairs complementary to strands of DNA and RNA.

Glycol nucleic acid (GNA) has a backbone composed of repeating glycol units linked by phosphodiester bonds. GNA shows a stronger Watson-Crick base pairing than DNA and RNA and requires a high temperature to melt a duplex GNA or GNA/DNA, GNA/RNA.

A “nucleic acid target” or “target nucleic acid” means a nucleic acid, or optionally a region thereof, that is to be detected. The target nucleic acid can have a nucleic acid sequence existing in the nature or any sequence designed and generated by human. For example, the nucleic acid sequence can be a genomic sequence of a prokaryotic or eukaryotic species. A prokaryotic species includes, for example, bacteria. A eukaryotic species includes, for example, a fungus, a plant, an animal, e.g., a mammal. In particular, the sequence of a target nucleic acid of interest can be found in public available databases, for example, the database of National Center for Biotechnology Information. The target nucleic acid can be single-stranded or double stranded. In certain embodiments, the target nucleic acid is a single stranded nucleotide polymer. In certain embodiments, the target nucleic acid is a single-stranded DNA or RNA (e.g., mRNA, siRNA, LncRNA). In certain embodiments, the target nucleic acid has 15 or more nucleotides, e.g., 20, 25, 30, 35, 40, 50, 60, 70, 80, 90, 100, 200, 300, 400, 500, 600, 700, 800, 900, 1000, 2000, 3000, 4000, 5000 or more nucleotides.

As used herein, a “nucleotide sequence” or “polynucleotide sequence” is a polymer of nucleotides (an oligonucleotide, a DNA, a nucleic acid, etc.) or a character string representing a nucleotide polymer, depending on context. From any specified nucleotide sequence, either the given nucleic acid or the complementary nucleic acid sequence can be determined.

A “label” as used herein is a moiety that facilitates detection of a molecule, typically by directly or indirectly providing a detectable signal. Common labels in the context of the present invention include fluorescent, luminescent, light-scattering, and/or colorimetric labels. Suitable labels include fluorophore and enzymes, such as Horse Radish Peroxidase (HRP), Alkaline Phosphatase (AP), as well as radionuclides, substrates, cofactros, inhibitors, chemiluminescent moieties, magnetic particles, and the like.

As used herein, a “label probe” refers to an entity that binds to a target molecule, directly or indirectly, and enables the target molecule to be detected, e.g., by a readout instrument. A label probe is typically a single-stranded polynucleotide that comprises one or more label that directed or indirectly provides a detectable signal. In certain embodiments, a label probe is a double-stranded polynucleotide comprising a label capable of providing detectable signal. The label can be covalently linked to the polynucleotide, or the polynucleotide can be configured to bind to the label (e.g., a biotinylated polynucleotide can bind a streptavidin associated label). The label probe can, for example, hybridize directly to a target nucleic acid, or it can hybridize to a nucleic acid (e.g., a target probe) that is in turn hybridized to the target nucleic acid or to one or more other nucleic acids that are hybridized to the nucleic acid. In preferred embodiments, the label probe can comprise a nucleotide sequence that is substantially complementary (e.g., at least 90% complementary, at least 95% complementary, at least 99% complementary, 100% complementary) to a nucleotide sequence (e.g., a gap nucleotide sequence) in a target probe, a bridge probe, or the like.

A “palindromic sequence” is a nucleotide sequence that is the same whether read 5′ to 3′ on one strand or 5′ to 3′ on the complementary strand with which it forms a double helix. For example, the DNA sequence 5′-AGCT-3′ is palindromic because its nucleotide-by-nucleotide complement is 3′-TCGA-5′, which gives the original sequence from 5′ to 3′.

As used herein, a “probe” is an entity that can be used in the detection of a target molecule. Typically, a probe in the present disclosure refers to a nucleic acid molecule, with or without modification. The probe can be both single-stranded and double-stranded nucleotide polymers. Unless indicated otherwise, the probes described in the present application is a single-stranded nucleotide polymer.

The term “sample” as used herein refers to any sample having or suspect of having the target nucleic acid, including sample of biological tissue or fluid origin, obtained, reached, or collected in vivo or in situ. Exemplary biological samples include but are not limited to cell lysate, a cell culture, a cell line, a tissue, an organ, a biological fluid, and the like. In certain embodiments, the sample is selected from the group consisting of sera, plasma, saliva, urine, cell and tissue.

The term “sequentially” means that the components (e.g., the head palindromic sequence, the gap sequence, and the tail palindromic sequence) in the bridge probe are juxtaposed in a 5′ end to 3′ end, or 3′ to 5′ order. For one example, the head palindromic sequence is located at the 5′ end of the bridge probe, the gap sequence is located in the downstream of the head palindromic sequence, and the tail palindromic sequence is located at the 3′ end of the bridge probe. It is understood that the bridge probe may include additional nucleotide sequence in adjacent to each component (e.g., the head palindromic sequence, the gap sequence, and the tail palindromic sequence) or between two components that does not interfere with the function of the bridge probe.

As used herein, a “target probe” refers to polynucleotide that is capable of hybridizing to a target nucleic acid and associating a label probe with the target nucleic acid. The target probe can hybridize to one or more nucleic acids (i.e., a bridge probe) that in turn hybridize to the label probe, or, in certain embodiments, it can hybridize directly to the label probe. The target probe thus includes a targeted nucleotide sequence (e.g., 6-20 nucleotides, 10-30 nucleotides, 12-25 nucleotides, 15-20 nucleotides) that is substantially complementary (e.g., at least 90% complementary, at least 95% complementary, at least 99% complementary, 100% complementary) to a targeting nucleotide sequence of the target nucleic acid and a pre-bridge nucleotide sequence (e.g., 6-20 nucleotides, 10-30 nucleotides, 12-25 nucleotides, 15-20 nucleotides) that is substantially complementary (e.g., at least 90% complementary, at least 95% complementary, at least 99% complementary, 100% complementary) to a tail nucleotide sequence of the bridge probe. In certain embodiments, the target probe further includes a third nucleotide sequence (e.g., the gap nucleotide sequence, e.g., 6-20 nucleotides, 10-30 nucleotides, 12-25 nucleotides, 15-20 nucleotides) that is substantially complementary (e.g., at least 90% complementary, at least 95% complementary, at least 99% complementary, 100% complementary) to a nucleotide sequence of the label probe. The target probe is preferably single-stranded.

### Ultra Sensitive Probe Composition

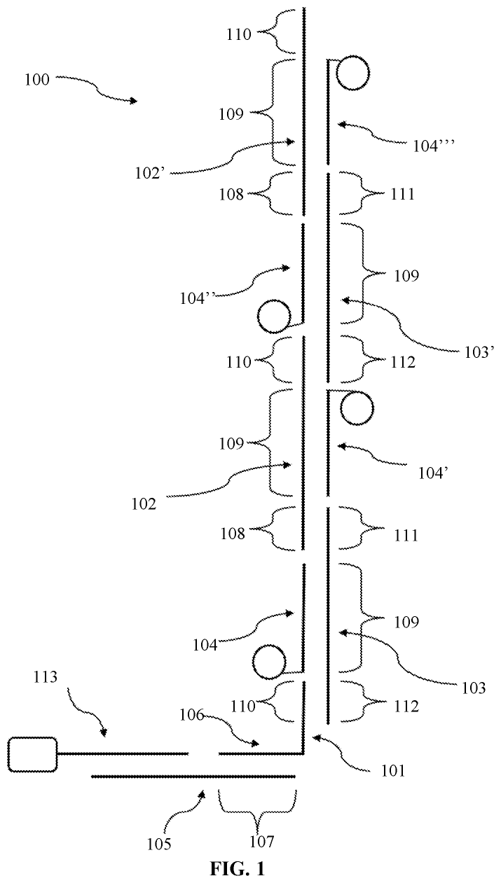

In one aspect, the present disclosure provides a probe composition with ultra sensitivity for detection of nucleic acid. An exemplary embodiment of ultra sensitive probe composition described herein is illustrated in FIG. 1. Referring to FIG. 1, the probe composition 100 is composed of a target probe 101, at least one first bridge probe 102 (two are shown), at least one second bridge probe 103 (two are shown), and label probes 104 (four are shown). In certain embodiments, each of the target probe, the first bridge probe and the second bridge probe are a single-stranded nucleotide polymer having 20-100 nucleotides, preferably 30-80 nucleotides, more preferably 40-60 nucleotides.

The target probe 101 includes sequentially (e.g., from 5′ to 3′) a targeting region having a targeting nucleotide sequence 106 and a tail region having a first tail nucleotide sequence 110 of 4-20 nucleotides long. In can be understood that there may be 0, 1, 2, 3, 4, 5, 6 or more nucleotides between the targeting region and the tail region. The targeting nucleotide sequence 106 is substantially complementary (e.g., at least 90% complementary, at least 95% complementary, at least 99% complementary, 100% complementary) to a targeted nucleotide sequence 107 of the target nucleic acid 105. Each first bridge probe 102 includes sequentially (e.g., from 5′ to 3′) a head region having a first head nucleotide sequence 108 of 6-20 nucleotides long, a gap region having a gap nucleotide sequence 109 of 6-20 nucleotides long, and a tail region having the first tail nucleotide sequence 110 of 4-20 nucleotides long. It can be understood that there may be 0, 1, 2, 3, 4, 5, 6 or more nucleotides between the head region and the gap region or between the gap region and the tail region. Each second bridge probe 103 includes sequentially (e.g., from 5′ to 3′) a head region having a second head nucleotide sequence 111 of 4-20 nucleotides long, which is substantially complementary (e.g., at least 90% complementary, at least 95% complementary, at least 99% complementary, 100% complementary) to the first head nucleotide sequence 108, a gap region having the gap nucleotide sequence 109, and a tail region having a second tail nucleotide sequence 112 of 4-20 nucleotides long, which is substantially complementary (e.g., at least 90% complementary, at least 95% complementary, at least 99% complementary, 100% complementary) to the first tail nucleotide sequence 110. It can be understood that there may be 0, 1, 2, 3, 4, 5, 6 or more nucleotides between the head region and the gap region or between the gap region and the tail region. Each label probe 104 includes a label nucleotide sequence of 6-20 nucleotides long and substantially complementary (e.g., at least 90% complementary, at least 95% complementary, at least 99% complementary, 100% complementary) to the gap nucleotide sequence 109, and is capable of hybridizing to the gap region.

When target probes 101, first bridge probes 102, second bridge probe 103 and label probes 104 are present in suitable solution and at suitable temperature (e.g., about 4° C. to about 50° C.), a complex is formed that can be used to detect a target nucleic acid 105. Referring to FIG. 1, a target probe 101 hybridizes to the target nucleic acid 105 through the hybridization of the targeting nucleotide sequence 106 and the targeted nucleotide sequence 107. At the same time, the target probe 101 hybridizes to a second bridge probe 103 through the hybridization of the first tail nucleotide sequence 110 and the second tail nucleotide sequence 112. The second bridge probe 103 further hybridizes to a first bridge probe 102 through the hybridization of the first head nucleotide sequence 108 and the second head nucleotide sequence 111. The second bridge probe 103 also hybridizes to a label probe 104 at the gap region. The first bridge probe 102 then further hybridizes to another second bridge probe 103′ through the hybridization of the first tail nucleotide sequence 110 and the second tail nucleotide sequence 112, and also hybridizes to a label probe 104′. The second bridge probe 103′ further hybridizes to another first bridge probe 102′ through the hybridization of the first head nucleotide sequence 108 and the second head nucleotide sequence 111. The second bridge probe 103′ also hybridizes to a label probe 104″ at the gap region. As such, the probe complex 100 can be formed through the hybridization chain reaction in which each bridge probe hybridizes to two other bridge probes at its head and tail region, respectively, and a label probe at the gap region. The probe complex 100 can include numerous label probes that generate signal strong enough to be able to detect target nucleic acid of low amount.

In certain embodiments, the ultra sensitive probe composition further includes a capture probe 113. The capture probe is capable to hybridize to the target nucleic acid 105 and includes a reagent that can be used to isolate, enrich or purify the target nucleic acid coupled to the ultra sensitive probe composition. In one embodiment, the capture probe is operably linked to a magnetic bead. In another embodiment, the capture probe is operably linked to a biotin, which can interact with a streptavidin-coupled bead.

In certain embodiments, a Yin-Yang probe (see U.S. Pat. No. 7,799,522, which is incorporated herein through reference) can be used as the label probe. A Yin-Yang probe is a double-stranded probe made of two complementary oligonucleotides of different lengths. One strand is labeled with a fluorophore and the other strand is labeled with a quencher. When self-hybridized in a stable double-stranded structure, the fluorophore and the quencher are in close proximity, thus the fluorophore is quenched by the quencher and the probe is non-fluorescent. In the presence of the target nucleic acid, the longer strand of the probe can spontaneously bind to the target nucleic acid, the double-stranded probe becomes dissociated, and the fluorophore become fluorescent. Using Yin-Yang probe can decrease background signal and further increase the sensitivity of the ultra sensitive composition disclosed herein.

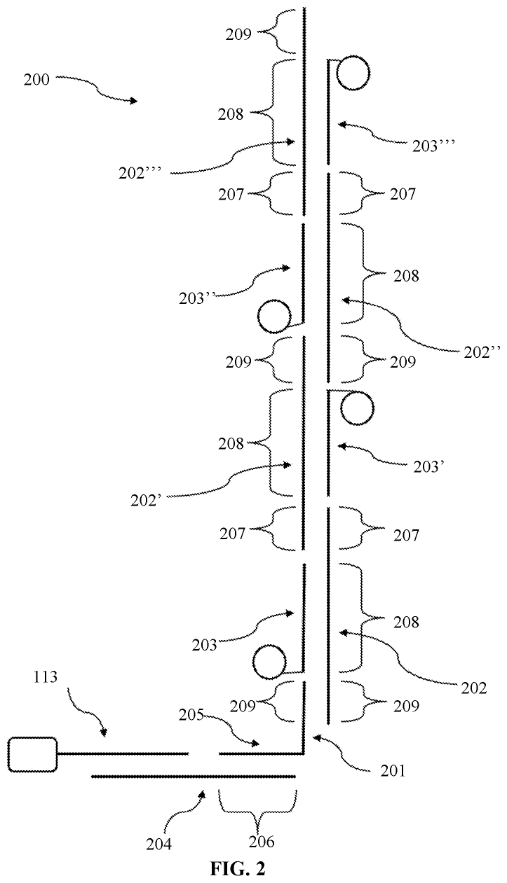

In certain embodiments, the first bridge probes and the second bridge probes as illustrated in FIG. 1 can be the same. FIG. 2 illustrates one of such embodiments. Referring to FIG. 2, the probe composition 200 is composed of target probe 201, bridge probes 202 (four are shown), and label probes 203 (four are shown). The target probe 201 includes sequentially (e.g., from 5′ to 3′) a targeting region having a targeting nucleotide sequence 205 and a tail region having a first tail nucleotide sequence 1. The targeting nucleotide sequence 205 is substantially complementary (e.g., at least 90% complementary, at least 95% complementary, at least 99% complementary, 100% complementary) to a targeted nucleotide sequence 206 of the target nucleic acid 204. The bridge probe 202 includes sequentially (e.g., from 5′ to 3′) a head region having a head nucleotide sequence 207, a gap region having a gap nucleotide sequence 208, and a tail region having a tail nucleotide sequence 209. Each of the head nucleotide sequence and the tail nucleotide sequence is independently a palindromic sequence. As a result, a head nucleotide sequence can hybridize to another head nucleotide sequence. Likewise, a tail nucleotide sequence can hybridize to another tail nucleotide sequence. Each label probe 203 includes a region having a nucleotide sequence substantially complementary (e.g., at least 90% complementary, at least 95% complementary, at least 99% complementary, 100% complementary) to the gap nucleotide sequence 208, and is capable of hybridizing to the bridge probe at the gap region.

When target probes 201, bridge probes 202 and babel probes 203 are present in suitable solution and at suitable temperature (e.g., about 4° C. to about 50° C.), a complex is spontaneously formed that can be used to detect a target nucleic acid 204. Referring to FIG. 2, a target probe 201 hybridizes to a target nucleic acid 204 through the hybridization of the targeted nucleotide sequence 206 and the targeting nucleotide sequence 205. The target probe 201 also hybridizes to a bridge probe 202 through the hybridization of two tail nucleotide sequences 209, one on the target probe 201, another on the bridge probe 202. The bridge probe 202 further hybridizes to another bridge probe 202′ through hybridization of the head nucleotide sequences 207. The bridge probe 202 also hybridizes to a label probe 203 at the gap region. The bridge probe 202′ further hybridizes to another bridge probe 202″ through hybridization of the head nucleotide sequences 207. The bridge probe 202′ also hybridizes to a label probe 203′ at the gap region. As such, a probe complex 200 can be formed through the chain reaction in which each bridge probe hybridizes to two other bridge probes at its head and tail region, respectively, and a label probe at the gap region. The probe complex can include numerous label probes that generate signal strong enough to be able to detect target nucleic acid of low amount.

The following examples are presented to illustrate the present invention. They are not intended to limiting in any manner.

### EXAMPLE 1

This example illustrates the detection of a target nucleic acid using a probe composition includes a target probe, a capture probe, a first bridge probe, a second bridge probe and a label probe.

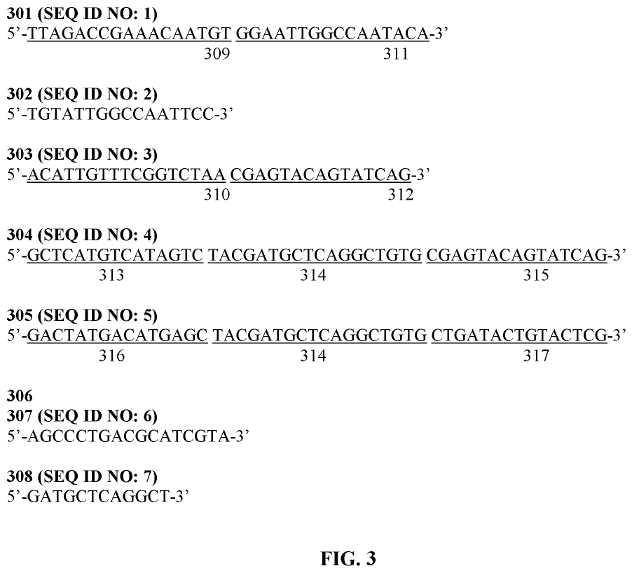

FIG. 3 shows the nucleotide sequence of the target nucleic acid 301, the capture probe 302 which is conjugated to a biotin at the 5′ end, the target probe 303, the first bridge probe 304, the second bridge probe 305 and the label probe 306. The target nucleic acid 301 contains a targeted nucleotide sequence 309 substantially complementary (e.g., at least 90% complementary, at least 95% complementary, at least 99% complementary, 100% complementary) to the targeting nucleotide sequence 310 in the target probe 303. The target nucleic acid 301 also contains a sequence 311 substantially complementary (e.g., at least 90% complementary, at least 95% complementary, at least 99% complementary, 100% complementary) to the sequence of the capture probe 302. The target probe 303 contains the targeting nucleotide sequence 310 and a tail nucleotide sequence 312. The first bridge probe 304 contains sequentially from 5′to 3′ a first head nucleotide sequence 313, a gap nucleotide sequence 314, and a first tail nucleotide sequence 315. The second bridge probe 305 contains a second head nucleotide sequence 316, a gap nucleotide sequence 314, and a second tail nucleotide sequence 317. The first head nucleotide sequence 313 is substantially complementary (e.g., at least 90% complementary, at least 95% complementary, at least 99% complementary, 100% complementary) to the second head nucleotide sequence 316. The first tail nucleotide sequence 315 is substantially complementary (e.g., at least 90% complementary, at least 95% complementary, at least 99% complementary, 100% complementary) to the second tail nucleotide sequence 317. The label probe 306 is a Yin-Yang probe consisting of a Yin probe 307 and a Yang probe 308. The Yang probe 307 is conjugated to a FAM fluorophore at the 5′ end, and the Yin probe 308 is conjugated to a fluorescence quencher Dabcyl at the 3′ end.

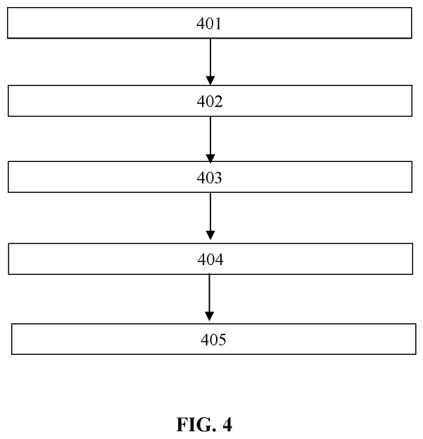

The process of the detection method is illustrated in FIG. 4. Referring to FIG. 4, in the first step 401, the target nucleic acid was mixed with the capture probe, the target probe, the first bridge probe, the second bridge probe and the label probe in a solution of 10 mM Tris, 15 mM MgCl₂. In the second step 402, the target nucleic acid, the capture probe, the target probe, the first bridge probes, the second bridge probes and the label probes then formed a complex. In the third step 403, the complex was pulled down by streptavidin coated magnetic beads (Nvigen Cat #K61002). In the step 404, the complex was then eluted from the Streptavidin coated magnetic beads by water/or a solution of 10 mM Tris, 15 mM MgCl₂, 95 degree boil for 5 min. In the step 405, the fluorescent signals from the eluted complex was then determined using a fluorescence reader (BioRad CFX96).

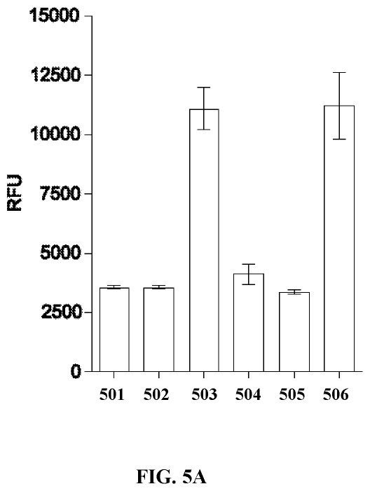

The result of the detection process is shown in FIGS. 5A and 5B. As shown in FIG. 5A, in the absence of the target nucleic acid 502, only background fluorescence was detected. In contrast, when the target nucleic acid was present 503, a strong fluorescence signal was detected. The detected fluorescence signal was specific to the target nucleic acid as confirmed by the result of using non-specific target probe 501, which gave rise to a signal comparable to the background. The result also showed that using single strand probe 506 generated a strong signal comparable to that using Yin-Yang probe 503. Columns 504 and 505 show the result using non-specific probe control and no target control (no single strand probe), respectively.

As shown in FIG. 5B, both the first and the second bridge probes are required for the formation of the probe composition as the presence of only one of the bridge probes gave rise to background signals. 511: non-specific probe control; 512: no target nucleic acid control; 513: target nucleic acid+first bridge probe 304+second bridge probe 305; 514: target nucleic acid+first bridge probe 304; 515: target nucleic acid+second bridge probe 305.

### EXAMPLE 2

This example illustrates the detection of a target nucleic acid using a probe composition includes a target probe, a capture probe, a bridge probe and a label probe.

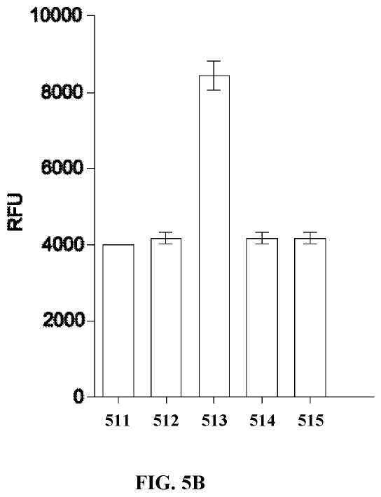

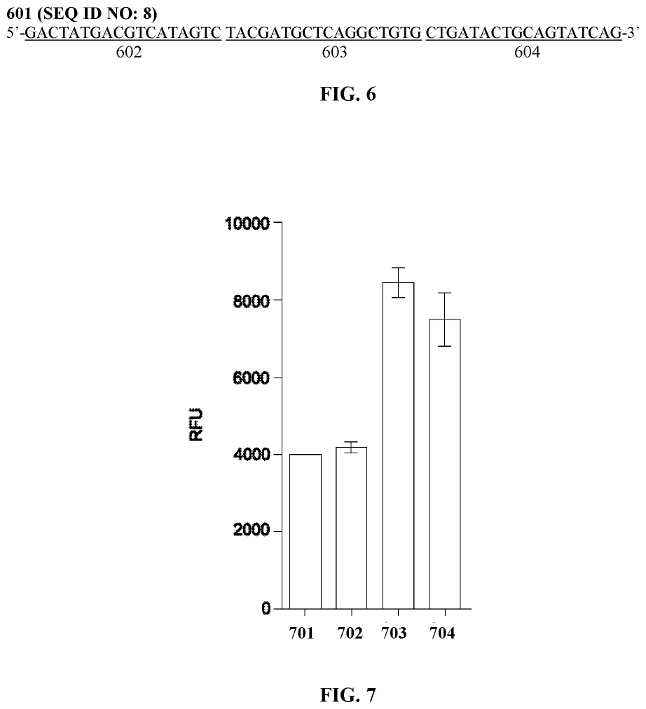

FIG. 6 shows the sequence of a bridge probe 601, which contains palindromic sequences at the head region 602 and the tail region 604. The bridge probe 601 also contains a gap region 603. The sequences of the target nucleic acid, the target probe, the capture probe and the label probe are the same as those used in Example 1.

The process of the detection was generally the same as Example 1.

FIG. 7 shows the detection result. As shown in FIG. 7, using O3 generates strong signal specific to the target nucleic acid. 701: non-specific probe control; 702: no target nucleic acid control; 703: target nucleic acid+first bridge probe 304+second bridge probe 305; 704: target nucleic acid+bridge probe 601.

### EXAMPLE 3

This example illustrates the detection of a target nucleic acid using a probe composition without a pull-down step.

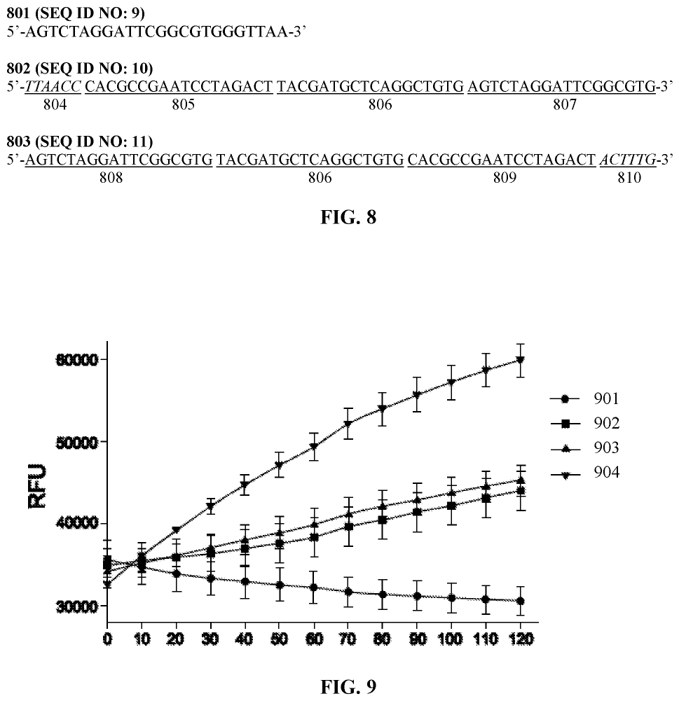

FIG. 8 shows the sequence of the target nucleic acid 801, the first bridge probe 802, and the second bridge probe 803. The label probe is the Yin-Yang probe 306 used in Example 1. The first bridge probe 802 contains a head nucleotide sequence 805, a gap nucleotide sequence 806, a tail nucleotide sequence 807 and six additional nucleotides 804 at the 5′ end of the head nucleotide sequence 805. The head nucleotide sequence 805 is substantially complementary (e.g., at least 90% complementary, at least 95% complementary, at least 99% complementary, 100% complementary) to the tail nucleotide sequence 807. The first bridge probe 802 forms a hairpin structure in the absence of the target nucleic acid 801 whereas the head nucleotide sequence 805 hybridizes to the tail nucleotide sequence 807. The second bridge probe 803 contains a head nucleotide sequence 808, the gap nucleotide sequence 806, a tail nucleotide sequence 809 and six additional nucleotides 810 at the 3′ end of the tail nucleotide sequence 809. The head nucleotide sequence 808 is substantially complementary (e.g., at least 90% complementary, at least 95% complementary, at least 99% complementary, 100% complementary) to the tail nucleotide sequence 809. The head nucleotide sequence 805 of the first bridge probe 802 is the same as the tail nucleotide sequence 809 of the second bridge probe 803. The six additional nucleotides 804 at the 5′ end of the first bridge probe 802 is substantially complementary (e.g., at least 90% complementary, at least 95% complementary, at least 99% complementary, 100% complementary) to the six nucleotides 810 at the 3′ end of the second bridge probe 803. The tail nucleotide sequence 807 of the first bridge probe 802 is the same as the head nucleotide sequence 808 of the second bridge probe 803. The second bridge probe 803 also forms a hairpin structure in the absence of the target nucleic acid 801 whereas the head nucleotide sequence 808 hybridizes to the tail nucleotide sequence 809.

In the presence of the target nucleic acid 801, which has a sequence substantially complementary (e.g., at least 90% complementary, at least 95% complementary, at least 99% complementary, 100% complementary) to the head nucleotide sequence 805 and the six additional nucleotides 804 of the first bridge probe 802, the hairpin structure of the first bridge probe 802 is opened up, releasing the tail nucleotide sequence 807 of the first bridge probe 802. The released the tail nucleotide sequence 807 of the first bridge probe 802 then hybridizes with the tail nucleotide sequence 809 of the second bridge probe 803, which opens up the hairpin structure of the second bridge probe 803 and releases the head nucleotide sequence 808 of the second bridge probe 803. The label probes then hybridize to the gap sequences of the first bridge probe 802 or the second bridge probe 803. This results a probe composition through a chain reaction.

As shown in FIG. 9, when the label probes were mixed with the first bridge probes and the second bridge probes, fluorescent signals were detected even in the absence of the target nucleic acid but in a background level. However, adding the target nucleic acid (40 nmol) initiated the hybridization chain reaction and opened up the hairpin structures of the first bridge probe and the second bridge probe for the binding of the label probe, which resulted an elevated fluorescent signal. 901: Probe only; 902: first bridge probe+second bridge probe+probe; 903: first bridge probe+second bridge probe+probe+target nucleic acid (4 nmol); 904: first bridge probe+second bridge probe+probe+target nucleic acid (40 nmol).

What is claimed is:

1. A composition comprising:
a) a target probe capable of hybridizing to a target nucleic acid, said target probe comprising a pre-bridge region having a first tail nucleotide sequence;
b) at least one first bridge probe, comprising sequentially
(i) a first head region having a first head nucleotide sequence;
(ii) a first gap region having a gap nucleotide sequence;
(iii) a first tail region having the first tail nucleotide sequence;
c) at least one second bridge probe, comprising sequentially
(i) a second head region having a second head nucleotide sequence complementary to the first head nucleotide sequence;
(ii) a second gap region having the gap nucleotide sequence;
(iii) a second tail region having a second tail nucleotide sequence complementary to the first tail nucleotide sequence; and
d) a label probe capable of hybridizing to the first and the second gap nucleotide region, said label probe comprising a label.

2. The composition of claim 1, wherein the first head nucleotide sequence consists of 4-20 nucleotides.

3. The composition of claim 1, wherein the first tail nucleotide sequence consists of 4-20 nucleotides.

4. The composition of claim 1, wherein the gap nucleotide sequence consists of 6-20 nucleotides.

5. The composition of claim 1, wherein the target probe and/or the first bridge probe and/or the second bridge probe and/or the label probe comprises at least one nucleotide analog.

6. The composition of claim 5, wherein the nucleotide analog is a locked nucleic acid nucleotide.

7. The composition of claim 1, wherein the label is a fluorophore, a horse radish peroxidase or an alkaline phosphatase.

8. The composition of claim 1, wherein the target nucleic acid is selected from the group consisting of a DNA, a cDNA, a RNA, a mRNA, a rRNA, a miRNA, a Lnc RNA and a siRNA.

9. The composition of claim 1, wherein the target probe further comprises a label region having the gap nucleotide sequence.

10. The composition of claim 1, further comprising a capture probe capable of hybridizing to the target nucleic acid, said capture probe comprising a magnetic bead or a biotin.

11. The composition of claim 1, wherein the first bridge probe is the same as the second bridge probe.

12. A composition comprising:
a) a target probe capable of hybridizing to a target nucleic acid, said target probe comprising a pre-bridge region having a tail palindromic sequence;
b) at least one bridge probe, wherein the bridge probe comprises sequentially
(i) a first region having a head palindromic sequence;
(ii) a second region having a gap sequence;
(iii) a third region having the tail palindromic sequence; and
c) a label probe capable of hybridizing to the bridge probe, said label probe comprising a label.

13. A method of detecting a target nucleic acid in a sample, said method comprising:
a) contacting a target probe to the target nucleic acid, wherein the target probe is capable of hybridizing to the target nucleic acid and comprises a pre-bridge region having a tail nucleotide sequence;
b) associating a first bridge probe, a second bridge probe and a label probe with the target probe, wherein:
the first bridge probe comprises sequentially
(i) a first head region having a first head nucleotide sequence;
(ii) a first gap region having a gap nucleotide sequence; and
(iii) a first tail region having the tail nucleotide sequence;
the second bridge probe comprises sequentially
(i) a second head region having a second head nucleotide sequence complementary to the first head nucleotide sequence;
(ii) a second gap region having the gap nucleotide sequence;
(iii) a second tail region having a second tail nucleotide sequence complementary to the first tail nucleotide sequence;
the label probe capable of hybridizing to the bridge probe, said label probe comprising a label; and
c) detecting the presence, absence, or amount of the label probe associated with the target molecule.

14. The method of claim 13, wherein the first head nucleotide sequence consists of 4-20 nucleotides.

15. The method of claim 13, wherein the first tail nucleotide sequence consists of 4-20 nucleotides.

16. The method of claim 13, wherein the gap nucleotide sequence consists of 6-20 nucleotides.

17. The method of claim 13, wherein the label is a fluorophore, a horse radish peroxidase or an alkaline phosphatase.

18. The method of claim 13, wherein the target nucleic acid is selected from the group consisting of a DNA, a cDNA, a RNA, a mRNA, a rRNA, a miRNA, a Lnc RNA and a siRNA.

19. The method of claim 13, wherein the target probe further comprises the gap nucleotide sequence.

20. The method of claim 13, further comprising the step of capturing said target nucleic acid with a capture probe capable of hybridizing to the target nucleic acid, said capture probe comprising a magnetic bead or a biotin.


SEQUENCE LISTING (11 sequences)

SEQ ID NO: 1 - DNA, 33 bp, Artificial Sequence
  Synthetic
ttagaccgaaacaatgtggaattggccaataca

SEQ ID NO: 2 - DNA, 16 bp, Artificial Sequence
  Synthetic
tgtattggccaattcc

SEQ ID NO: 3 - DNA, 32 bp, Artificial Sequence
  Synthetic
acattgtttcggtctaacgagtacagtatcag

SEQ ID NO: 4 - DNA, 48 bp, Artificial Sequence
  Synthetic
gctcatgtcatagtctacgatgctcaggctgtgcgagtacagtatcag

SEQ ID NO: 5 - DNA, 48 bp, Artificial Sequence
  Synthetic
gactatgacatgagctacgatgctcaggctgtgctgatactgtactcg

SEQ ID NO: 6 - DNA, 17 bp, Artificial Sequence
  Synthetic
agccctgacgcatcgta

SEQ ID NO: 7 - DNA, 12 bp, Artificial Sequence
  Synthetic
gatgctcaggct

SEQ ID NO: 8 - DNA, 54 bp, Artificial Sequence
  Synthetic
gactatgacgtcatagtctacgatgctcaggctgtgctgatactgcagtatcag

SEQ ID NO: 9 - DNA, 24 bp, Artificial Sequence
  Synthetic
agtctaggattcggcgtgggttaa

SEQ ID NO: 10 - DNA, 60 bp, Artificial Sequence
  Synthetic
ttaacccacgccgaatcctagacttacgatgctcaggctgtgagtctaggattcggcgtg

SEQ ID NO: 11 - DNA, 60 bp, Artificial Sequence
  Synthetic
agtctaggattcggcgtgtacgatgctcaggctgtgcacgccgaatcctagactactttg